(19) United States
(12) Patent Application Publication
(10) Pub. No.: US 2020/0323656 A1
(43) Pub. Date: Oct. 15, 2020

(54) MONOCENTRIC PROSTHETIC KNEE

(71) Applicant: EMPOWERED TECHNOLOGIES, LLC, Newark, DE (US)

(72) Inventors: Andrew DIRKS, Crownsville, MD (US); Claire PADDOCK, Washington, MO (US); Sean WALKER, Newark, DE (US); John HORNE, Wilmington, DE (US)

(21) Appl. No.: 16/956,434

(22) PCT Filed: Dec. 20, 2018
(86) PCT No.: PCT/US18/66789

§ 371 Date: Jun. 19, 2020

Related U.S. Application Data
(60) Provisional application No. 62/608,886, filed on Dec. 21, 2017.

(51) Int. Cl. A61F 2/64

(52) U.S. Cl. CPC A61F 2/64; A61F 2002/6854

(57) ABSTRACT

A prosthetic knee (10) for a transfemoral amputee has a monocentric rotational joint (20) that rotatably attaches an upper housing (12) attachable to the amputee's residual limb to a lower housing (16) attachable to a below-the knee prosthesis. A locking mechanism (32) disposed in the lower housing (16) has a piston (34) biased in a locked position releasably engaging the upper housing (12) preventing rotation of the rotational joint (20). An unlocking mechanism (32) having a trigger (48) attached to a posterior side of the lower housing (16) by a pair of spaced-apart trigger links (52) is configured to unlock the locking mechanism (32). A piston rod (62) having a first piston-rod end (64) pivotably attached to one of the trigger links (52) has a second piston-rod end (68) pivotably attached to the piston (34). A force applied to the trigger (48) causes the first piston-rod end (64) to pivot counterclockwise driving the piston rod (62) downwardly disengaging the piston (34) from the locked position, allowing rotation of the rotational joint (20).

### CROSS-REFERENCE TO RELATED APPLICATIONS

This application claims the benefit of U.S. Provisional Patent Application No. 62/608,886, filed Dec. 21, 2018, the disclosures of which are incorporated herein by reference.

### BACKGROUND OF THE INVENTION

The present invention relates to a monocentric prosthetic knee for a transfemoral amputee and more specifically to a monocentric prosthetic knee having an unlocking mechanism.

A wide range of technologies are employed to service the varying needs of a transfemoral amputee. The basic requirements for a prosthetic leg for a transfemoral amputee include stability and mechanical compatibility both while standing and during the different phases of stance while walking (or running). Tradeoffs exist between security and stability (balance behavior) and walking or running performance (dynamic behavior). For lower activity amputees who have an increased fall risk, such as the elderly, frail, or new amputees, it is imperative that they are outfit with a prosthetic device that emphasizes balance behavior features to prevent catastrophic injuries resultant of a fall.

Traditionally, prosthetic legs with manual locking knees have been provided as the safest option for patients with weak musculature or balances issues. The knees of these prosthetic legs do not bend until a release mechanism is operated to free a knee lock. The safety provided by these knees is reduced by requiring the patient, who will typically ambulate using a walker or cane, to remove their hand from their assistive walking device in order to activate the unlocking mechanism.

Accordingly. there is a need for a prosthetic leg with a manual locking knee that provides the benefits of stability and security as existing devices while allowing for hands-free unlocking.

### BRIEF SUMMARY OF THE INVENTION

Briefly stated, one embodiment of the present invention is directed to a monocentric prosthetic knee for a transfemoral amputee. The monocentric prosthetic knee comprises an upper housing having a prosthetic socket connector configured to attached the upper housing to a residual limb of the transfemoral amputee and a lower housing having an adapter configured to attach the lower housing to a below-the knee prosthesis. A monocentric rotational joint rotatably attaches the upper housing to the lower housing. The monocentric rotational joint has a monocentric rotational joint axis of rotation. A locking mechanism is disposed in the lower housing. The locking mechanism has a piston biased in a locked position releasably engaging the upper housing preventing rotation of the rotational joint. An unlocking mechanism is operatively coupled to the locking mechanism and is configured to unlock the locking mechanism. The unlocking mechanism comprises a trigger attached to a posterior side of the lower housing by a pair of spaced-apart trigger links. Each trigger link has a first trigger-link end pivotably attached to the posterior side of the lower housing and that is pivotable about a first trigger-link end axis and a second trigger-link end pivotably attached to the trigger and that is pivotable about a second trigger-link end axis. A piston rod having a first piston-rod end pivotably attached to the trigger is pivotable about a first piston-rod end axis and has a second piston-rod end pivotably attached to the piston. A force applied to the trigger pivots the pair of trigger links clockwise about the first and second trigger-link axes, which, in turn, causes the first piston-rod end to pivot counterclockwise about the first piston-rod axis driving the piston rod downwardly disengaging the piston from the locked position and allowing rotation of the rotational joint.

### BRIEF DESCRIPTION OF THE SEVERAL VIEWS OF THE DRAWINGS

The detailed description of the invention, will be better understood when read in conjunction with the appended drawings. For the purpose of illustrating the invention, there is shown in the drawings an embodiment which is presently preferred. It should be understood, however, that the invention is not limited to the precise arrangements and instrumentalities shown.

In the drawings:

FIG. 1. is a side, elevational view of a prosthetic knee in a locked position in accordance with a preferred embodiment of the present invention;

FIG. 2. is a side, elevational view of the prosthetic knee of FIG. 1 unlocked and in flexion;

FIG. 3. is an exploded view of upper and lower housing of the prosthetic system of FIG. 1;

FIG. 4. is an exploded view of an unlocking mechanism removed from the prosthetic system of FIG. 1;

FIG. 5. is a side, elevational view of the piston of FIG. 1; and

FIG. 6. is a side, elevational view of the piston rod of FIG. 1.

### DETAILED DESCRIPTION OF THE INVENTION

Reference will now be made in detail to an embodiment of the invention, examples of which are illustrated in the accompanying drawings. The terminology used in the description of the invention herein is for the purpose of describing the particular embodiment only and is not intended to be limiting of the invention.

As used in the description of the invention, the singular forms “a”, “an” and “the” are intended to include the plural forms as well, unless the context clearly indicates otherwise. The words “and/or” as used herein refers to and encompasses any and all possible combinations of one or more of the associated listed items. The words “comprises” and/or “comprising,” when used in this specification, specify the presence of the stated features, integers, operations, elements, and/or components, but do not preclude the addition of one or more other features, integers, steps, operations, elements, components, and/or groups thereof.

It should also be understood that the terms “about,” “approximately,” “generally,” “substantially” and like terms, used herein when referring to a dimension or characteristic of a component of the invention, indicate that the described dimension/characteristic is not a strict boundary or parameter and does not exclude minor variations therefrom that are functionally similar. At a minimum, such references that include a numerical parameter would include variations that, using mathematical and industrial principles accepted in the art (e.g., rounding, measurement or other systematic errors, manufacturing tolerances, etc.), would not vary the least significant digit.

The words “right,” left,” “lower,” “upper,” “front” and “rear” designate directions in the drawings to which reference is made. The words “inwardly” and “outwardly” refer to directions toward and away from, respectively, the geometric center of the hinge, and designated parts thereof. The terminology includes the words noted above, derivatives thereof and words of similar import.

Although the words first, second, etc., are used herein to describe various elements, these elements should not be limited by these words. These words are only used to distinguish one element from another. For example, a first link could be termed a second link without departing from the scope of the present invention.

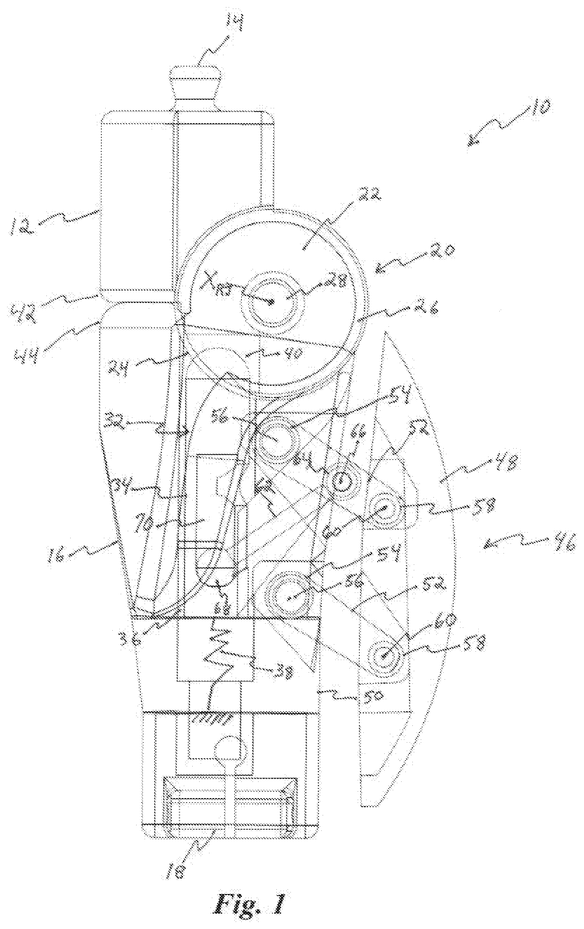

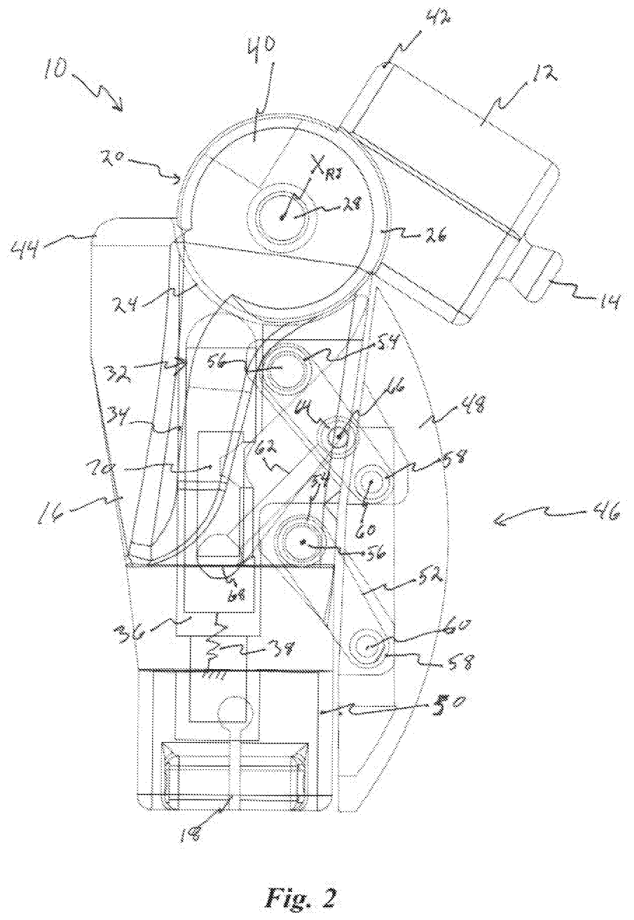

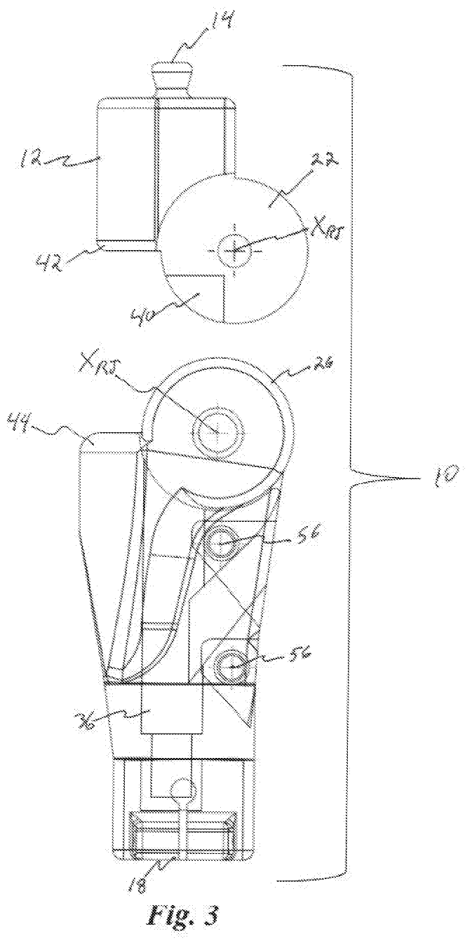

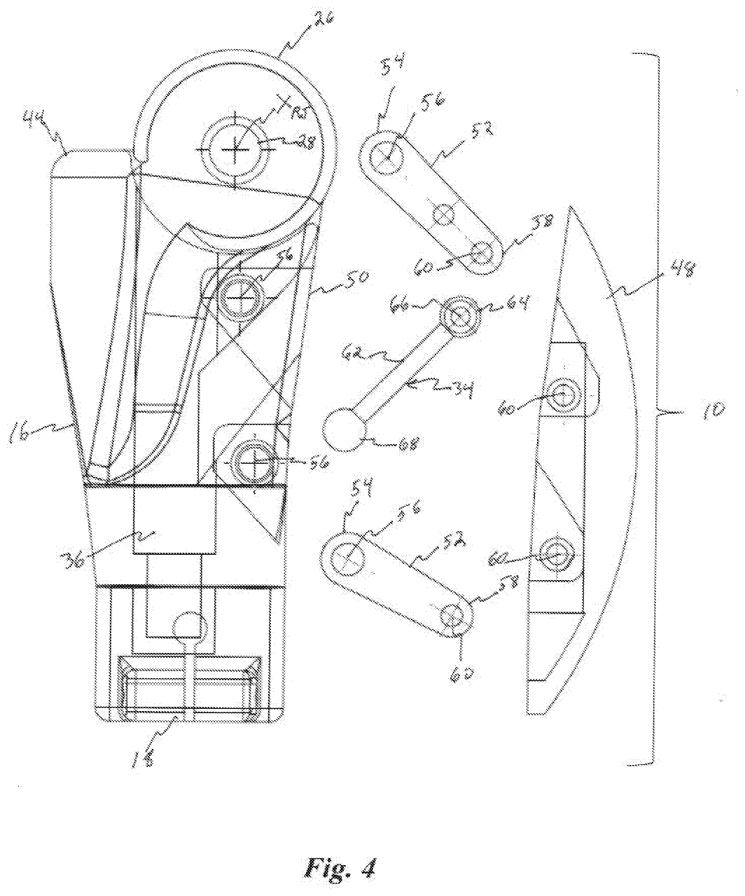

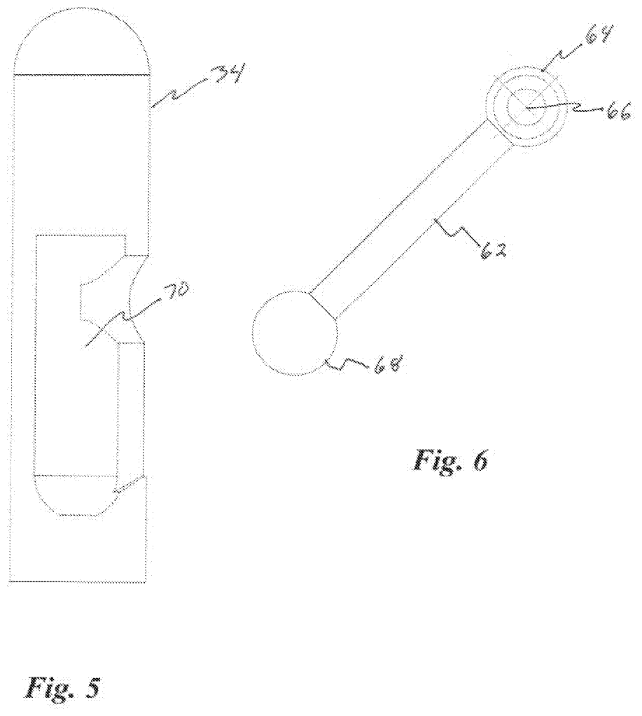

Referring to the drawings in detail, where like numerals indicate like elements throughout, there is shown in FIGS. 1-6 a first preferred embodiment of a monocentric prosthetic knee, generally designated 10, and hereinafter referred to as the “knee” 10 in accordance with the present invention.

The knee 10 comprises an upper housing 12 having a prosthetic socket connector 14 configured to attached the upper housing 12 to a residual limb of the transfemoral amputee. The knee 10 also comprises a lower housing 16 having an adapter 18 configured to attach the lower housing 16 to a below-the knee prosthesis.

A monocentric rotational joint 20 rotatably attaches the upper housing 12 to the lower housing 16. The monocentric rotational joint 20 has a monocentric rotational-joint axis of rotation X_{RJ}. The upper housing 12 comprises an upper housing cylinder 22 that forms an inner part of the rotational joint 20. The lower housing 16 has a lower housing well 24 in which the rotational joint 20 is disposed and an outer edge 26 that contains the rotational joint 20 in the lower housing well 24. A rotational joint axle 28 about which the upper housing cylinder 22 rotates is coincident with the rotational joint axis X_{RJ}.

In some embodiments, in addition to the rotary joint 20, resistive bands 30 may operatively couple the upper housing 12 to the lower housing 16. The resistive bands 30 are preferably an elastomeric material that may serve to slow the rotation of the rotational joint 20 allowing slow decent as an amputee attempts to sit from a standing position and alternatively, as an extension assist system when the amputee stands from the sitting position.

A locking mechanism 32 is disposed in the lower housing 16. The locking mechanism 32 has a piston 34 biased in a locked position releasably engaging the upper housing 12 preventing rotation of the rotational joint 20. The lower housing 16 has a lower housing bore 36 that extends downwardly in a center of the lower housing well 24. The piston 34 is movably disposed in the lower housing bore 36. A compression spring 38 in the lower housing bore 36 biases the piston 34 upwardly. The upper housing cylinder 22 has an upper housing cylinder detent 40 extending radially inwardly. When the locking mechanism 40 is in the locked position, a portion of the piston 34 extends into the upper housing cylinder detent 40.

In some embodiments, the upper housing 12 has an anterior upper housing portion 42 that abuts a corresponding anterior lower housing portion 44 preventing hyperextension when the locking mechanism 32 is in the locked position. In other embodiments, a plurality of upper housing cylinder detents 40 are located along a circumference of the upper housing cylinder 22 and are configured to receive the piston 34 stepwise locking the rotational joint 20 and therefore the monocentric prosthetic knee 10 in a succession of angular positions.

An unlocking mechanism 46 is operatively coupled to the locking mechanism 40 and is configured to unlock the locking mechanism 46. The unlocking mechanism 46 comprises a trigger 48 attached to a posterior side 50 of the lower housing 16 by a pair of spaced-apart trigger links 52. Each trigger link 52 has a first trigger-link end 54 pivotably attached to the posterior side 50 of the lower housing 16. The first trigger-link end 54 is pivotable about a first trigger-link end axis 56. Each trigger link 52 also has a second trigger-link end 58 spaced from the first trigger link end 54. The second trigger link end is pivotably attached to the trigger 48. The second trigger-link end 58 is pivotable about a second trigger-link end axis 60.

A piston rod 62 has a first piston-rod end 64 pivotably attached to the trigger 48 between the first trigger-link end 54 and the second trigger link end 58 of one trigger link of the pair of trigger links 52. The first piston-rod end 64 is pivotable about a first piston-rod end axis 66. The piston rod 62 also has a second piston-rod end 68 spaced from the first piston rod end 64 and pivotably attached to the piston 34. Preferably, the piston 34 has a piston cavity 70 and the second piston-rod end 68 has the shape of a sphere and is movably disposed in the piston cavity 70.

In use, when the rotational joint 20 is in the locked position, the rotational joint 20 may be unlocked by applying a force to the trigger 48 which in reaction thereto pivots the pair of trigger links 52 clockwise about the first and second trigger-link axes 56, 69, which, in turn, causes the first piston-rod end 64 to pivot counterclockwise about the first piston-rod axis 66 driving the piston rod 62 downwardly disengaging the piston 34 from the locked position and allowing rotation of the rotational joint 20. Removal of the force allows the piston 34, under the biasing of the compression spring 38 to move upwardly causing a portion of the piston 34 to extend into the upper housing cylinder detent 40 preventing rotation of the rotational joint 20.

Preferably, the unlocking force is applied to the trigger 28 through contact with an abutting structure, such as the front surface of a chair; alternatively, the unlocking force may be applied to the trigger 28 by the amputee's hand.

The foregoing detailed description of the invention has been disclosed with reference to specific embodiments. However, the disclosure is not intended to be exhaustive or to limit the invention to the precise forms disclosed. Those skilled in the art will appreciate that changes could be made to the embodiments described above without departing from the broad inventive concept thereof. Therefore, the disclosure is intended to cover modifications within the spirit and scope of the present invention as defined by the appended claims.

All references, patent applications, and patents mentioned above are incorporated herein by reference in their entirety and are not to be construed as an admission that any of the cited documents constitutes prior art, or as an admission against interest in any manner.

We claim:

1. A monocentric prosthetic knee (10) for a transfemoral amputee, the monocentric prosthetic knee (10) comprising:
an upper housing (12) having a prosthetic socket connector (14) configured to attached the upper housing (12) to a residual limb of the transfemoral amputee;
a lower housing (16) having an adapter (18) configured to attach the lower housing (16) to a below-the knee prosthesis;
a monocentric rotational joint (20) rotatably attaching the upper housing (12) to the lower housing (16), the monocentric rotational joint (20) having a monocentric rotational joint axis of rotation (X_{RJ});
a locking mechanism (32) disposed in the lower housing (16), the locking mechanism (32) having a piston (34) biased in a locked position releasably engaging the upper housing (12) preventing rotation of the rotational joint (20); and
an unlocking mechanism (46) operatively coupled to the locking mechanism (32) and configured to unlock the locking mechanism (32),
characterized in that the unlocking mechanism (46) comprises:
a trigger (48) attached to a posterior side of the lower housing (16) by a pair of spaced-apart trigger links (52), each trigger link (52) having a first trigger-link end (54) pivotably attached to the posterior side of the lower housing (16) and pivotable about a first trigger-link end axis (56) and a second trigger-link end (58) pivotably attached to the trigger (48) and pivotable about a second trigger-link end axis (60); and
a piston rod (62) having a first piston-rod end (66) pivotably attached to the trigger (48) and pivotable about a first piston-rod end axis (66) and a second piston-rod end (68) pivotably attached to the piston (34),
wherein a force applied to the trigger (48) pivots the pair of trigger links (52) clockwise about the first and second trigger-link axes (56, 60), which, in turn, causes the first piston-rod end (64) to pivot counterclockwise about the first piston-rod axis (66) driving the piston rod (62) downwardly disengaging the piston (34) from the locked position allowing rotation of the rotational joint (20).

2. The monocentric prosthetic knee according to claim 1, wherein:
the upper housing comprises an upper housing cylinder that forms an inner part of the rotational joint,
the lower housing has a lower housing well in which the rotational joint is disposed and an outer edge that contains the rotational joint, and
an rotational joint axle is coincident with the rotational joint axis.

3. The monocentric prosthetic knee according to claim 2, wherein:
the upper housing cylinder has an upper housing cylinder detent extending radially inwardly;
a lower housing bore extends downwardly in a center of the lower housing well;
the piston is movably disposed in the lower housing bore;
a compression spring in the bore biases the piston upwardly; and
a portion of the piston is in the upper housing cylinder detent when the locking mechanism is in the locked position.

4. The monocentric prosthetic knee according to claim 1, wherein:
the piston has a piston cavity; and
the second piston-rod end has the shape of a sphere and is movably disposed in the piston cavity.

5. The monocentric prosthetic knee according to claim 1, further comprising resistive bands operatively coupling the upper housing to the lower housing.

6. The monocentric prosthetic knee according to claim 1, wherein:
an anterior upper housing portion abuts a corresponding anterior lower housing portion preventing hyperextension when the locking mechanism is in the locked position.

7. The monocentric prosthetic knee according to claim 1, wherein:
a plurality of upper housing cylinder detents are located along a circumference of the upper housing cylinder and configured to receive the piston stepwise locking the monocentric prosthetic knee in a succession for angular positions.
